(19) United States
(12) Patent Application Publication
(10) Pub. No.: US 2016/0374566 A1
(43) Pub. Date: Dec. 29, 2016

(54) SAMPLE-COUNT-BASED SENSOR DATA CALCULATIONS

(71) Applicant: Microsoft Technology Licensing, LLC, Redmond, WA (US)

(72) Inventors: Han Yee Mimi Fung, Bellevue, WA (US); Haithem Albadawi, Redmond, WA (US)

(21) Appl. No.: 14/748,030

(22) Filed: Jun. 23, 2015

(51) Int. Cl. A61B 5/0205; A61B 5/00; A61B 5/11

(52) U.S. Cl. CPC A61B 5/0205; A61B 5/11; A61B 5/681

(57) ABSTRACT

A computing device includes a time-dependent sensor configured to output a time-dependent datum at a time-dependent frequency, the time-dependent datum specifying a sensed parameter. A time-independent, sample-count-based calculation module is configured to output a new time-agnostic datum after receiving a specific number of the time-dependent data regardless of elapsed time taken to receive the specific number of the time-dependent data.

### BACKGROUND

Sensors may utilize a real-time clock to sample and output data at various time-dependent frequencies. Computing devices may receive and utilize such sensor data to determine parameters. After receiving such data over a period of time, the computing devices may analyze the data collected over such period.

### BRIEF DESCRIPTION OF THE DRAWINGS

FIGS. 1A and 1B show a wearable computing device.

FIG. 2 schematically shows example sensors and a computing system that may be included in the wearable computing device of FIGS. 1A and 1B.

FIGS. 3-6 show examples of sensors, sample-count-based calculation modules and corresponding data flow in different use cases.

FIG. 7 shows a method of updating a time-agnostic biometric metric.

FIG. 8 schematically shows a sensory and logic system useable to output a new time-agnostic datum after receiving a number of time-dependent data.

### DETAILED DESCRIPTION

The present disclosure is directed to time-independent, sample-count-based calculation modules that output a time-agnostic datum, such as a biometric metric, after receiving a specific number of time-dependent data from a time-dependent sensor. For example, a time-dependent sensor may be configured to output a time-dependent datum at a time-dependent frequency, with the time-dependent datum specifying a sensed parameter. A time-independent, sample-count-based calculation module may be configured to output a new time-agnostic datum after receiving a specific number of the time-dependent data regardless of elapsed time taken to receive the specific number of the time-dependent data.

While described below in the context of a wearable computing device, it is to be understood that the principles of the present disclosure may be used in numerous different applications, and with various different types of sensory-and-logic systems. Additionally, while the examples below describe various time-agnostic data as biometric metrics, it will be appreciated that the principles of the present disclosure encompass time-agnostic data in various other forms, such as device performance data, environmental condition data, etc.

In various applications, computing devices may gather data collected by time-dependent sensors that operate at a given frequency governed by a real-time clock. For example, an inertial measuring unit (IMU) may measure and output accelerations at 60 Hz. or any suitable frequency. A computing device may collect and utilize the output to determine a corresponding value. For example, a running watch with an IMU comprising an accelerometer may utilize a time-dependent distance algorithm to analyze accelerations collected over a period of time and determine a distance traveled. As used herein, “time-dependent” means that a device or process generates, receives and/or transforms data according to a temporally specific schedule over an elapsed time. For example, a time-dependent accelerometer may generate and output acceleration measurements exactly once every 16.6 milliseconds for a one hour period. As such, the time-dependent accelerometer will generate and output exactly 216,000 acceleration measurements in a one hour period.

In the present example, the time-dependent distance algorithm may receive acceleration data at the same temporally specific schedule as the IMU outputs such data. In other words, the time-dependent distance algorithm must receive 216,000 acceleration measurements over a one hour period. Correspondingly, the time-dependent distance algorithm may determine a cumulative distance traveled over the period of time during which such data is collected.

In one example, the time-dependent distance algorithm may collect acceleration data over a one year period. In some examples, a user may desire to validate the accuracy of the distance algorithm with respect to data collected over such a one year period. However, because the distance algorithm is time-dependent, one year would be required to elapse before the accuracy of the distance algorithm could be evaluated.

To accelerate such a validation process and provide flexibility in data sampling rates utilized by such algorithms, a time-independent, sample-count-based calculation module may be utilized according to the present disclosure. For example, as described in more detail below, a time-independent, sample-count-based calculation module may be configured to output a new time-agnostic datum after receiving a specific number of time-dependent data from a time-dependent sensor, regardless of the elapsed time taken to receive such time-dependent data. In other words, the time-independent, sample-count-based calculation module is not programmed to operate at any temporally specific schedule. Instead, the time-independent, sample-count-based calculation module may dynamically adjust its output schedule in proportion to whatever input schedule is currently being used to feed the module.

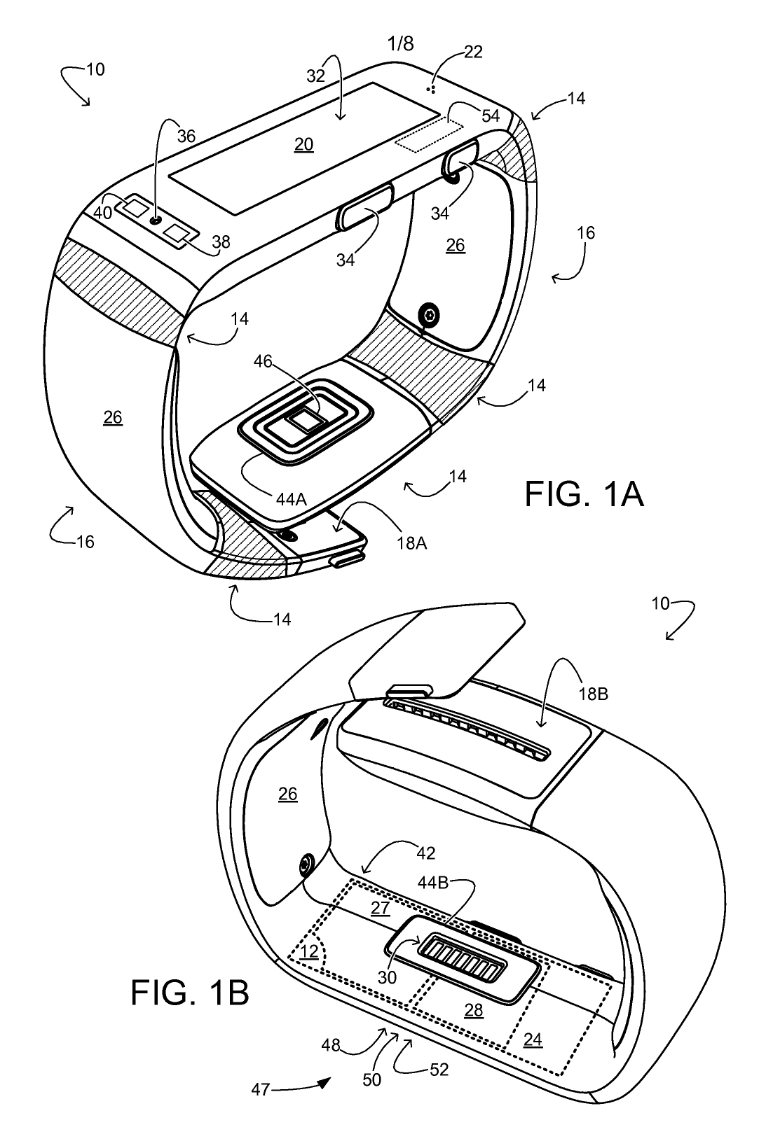

FIGS. 1A and 1B show aspects of an example sensory-and-logic system in the form of a wearable computing device 10 that includes a computing system 12 comprising one or more time-independent, sample-count-based calculation modules according to the present disclosure. The illustrated device is band-shaped, with at least four flexion regions 14 linking less flexible regions 16. The flexion regions may be elastomeric in some examples. Fastening componentry 18A and 18B is arranged at both ends of the device.

The flexion regions and fastening componentry enable the device to be closed into a loop and to be worn on a user's wrist. In other implementations, wearable computing devices of a more elongate band shape may be worn around the user's bicep, waist, chest, ankle, leg, head, or other body part. The device, for example, may take the form of eye glasses, a head band, an arm-band, an ankle band, a chest strap, or an implantable device to be implanted in tissue.

Wearable computing device 10 may include various functional components integrated into regions 16. For example, the wearable computing device 10 may include the computing system 12, display 20, loudspeaker 22, and communication suite 24. The wearable computing device 10 also includes various sensors, such as an optical heart rate sensor 46 and motion sensing componentry. In some examples, the motion sensing componentry may comprise an inertial measuring unit (IMU) 47. In the example of FIGS. 1A and 1B such inertial measuring unit 47 comprises an accelerometer 48, gyroscope 50, and magnetometer 52. In other examples, the motion sensing componentry may comprise any combination of accelerometers, gyroscopes and magnetometers.

One or more of such sensors may be time-dependent. As described above, such a time-dependent sensor may be configured to output a time-dependent datum at a time-dependent frequency, where the time-dependent datum specifies a sensed parameter. For example, a time-dependent accelerometer 48 may be configured to output a time-dependent acceleration at a given frequency, such as 60 Hz. It will be appreciated that any suitable time-dependent frequency may be utilized.

In some examples the accelerometer and gyroscope may furnish inertial and/or rotation rate data along three orthogonal axes as well as rotational data about the three axes, for a combined six degrees of freedom (6DOF). This 6DOF sensory data may be used to provide pedometer and calorie-counting functions, for example.

Data from the accelerometer and gyroscope may be combined with geomagnetic data from the magnetometer to further define the inertial and rotational data in terms of geographic orientation. The wearable computing device also may include a global positioning system (GPS) receiver 54 for determining the wearer's geographic location and velocity. In some configurations, the antenna of the GPS receiver may be relatively flexible and extend into flexion regions 14.

The above-described components may draw power from one or more energy-storage cells 26. A battery—e.g., a lithium ion battery—is one type of energy-storage cell suitable for this purpose. Examples of alternative energy-storage cells include super- and ultra-capacitors. In devices worn on the user's wrist, the energy-storage cells may be curved to fit the wrist, as shown in the drawings.

In general, energy-storage cells 26 may be replaceable and/or rechargeable. In some examples, recharge power may be provided through a universal serial bus (USB) port 30, which includes a magnetic latch to secure a complementary USB connector. In other examples, the energy storage cells may be recharged by wireless inductive or ambient-light charging. In still other examples, the wearable computing device may include electro-mechanical componentry to recharge the energy storage cells from the user's adventitious or purposeful body motion. For example, batteries or capacitors may be charged via an electromechanical generator integrated into wearable computing device 10. The generator may be turned by a mechanical armature that turns while the user is moving and wearing wearable computing device 10.

The optical heart rate sensor 46 may comprise an optical source configured to illuminate one or more blood vessels through a user's skin, an optical sensor configured to measure reflected illumination from the blood vessels, and one or more energy storage cells. The optical heart rate sensor 46 may further comprise a storage machine holding instructions executable by a logic machine to operate the optical source and optical sensor at varying rates of energy consumption during different conditions and/or timeframes. By dynamically changing the rate of energy consumption, the optical heart rate sensor 46 may consume less power during times of low user activity or for non-critical applications.

In some configurations, an optical heart rate sensor 46 may be positioned within a wearable computing device 10 such that an optical source illuminates a radial artery through the skin of the user while the wearable computing device is worn by the user. Alternatively, an optical heart rate sensor 46 and its associated computing system may be housed separately and configured to communicate via a communication suite. For example, an optical heart rate sensor 46 may be included in a head set and configured to illuminate capillaries located in the user's ear lobe while the head set is worn by the user. An optical sensor may be configured to sense light reflected off of blood vessels located beneath the skin of the user (e.g., wrist worn), or the optical sensor may be configured to sense light transmitted through blood vessels located beneath the skin of the user (e.g., ear worn).

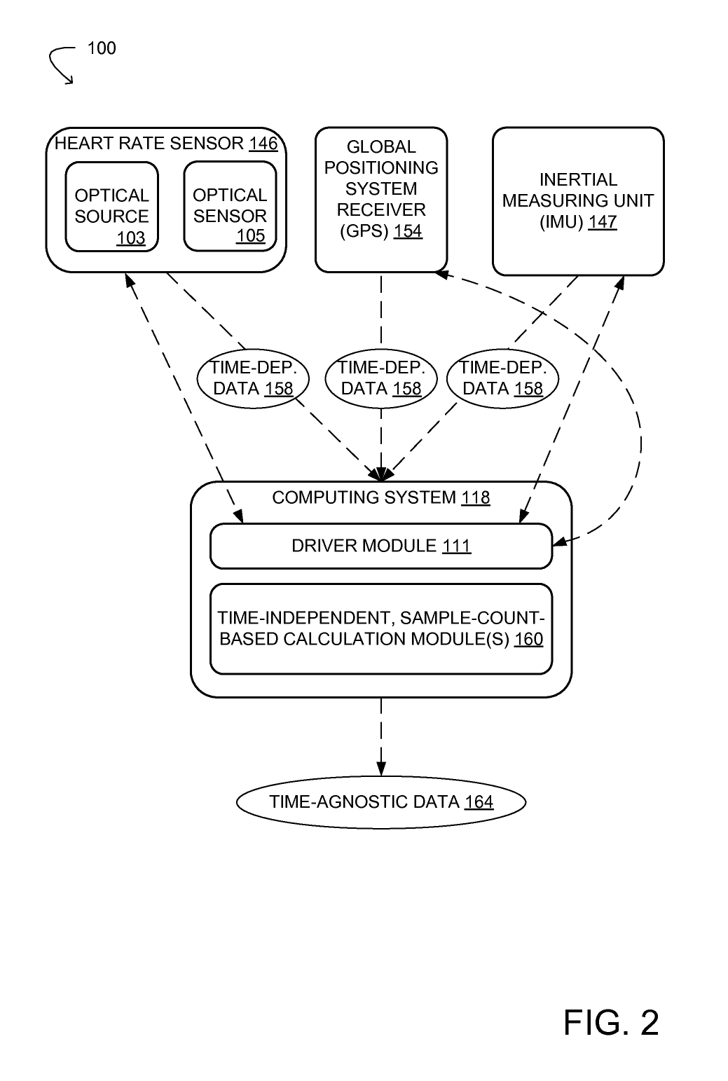

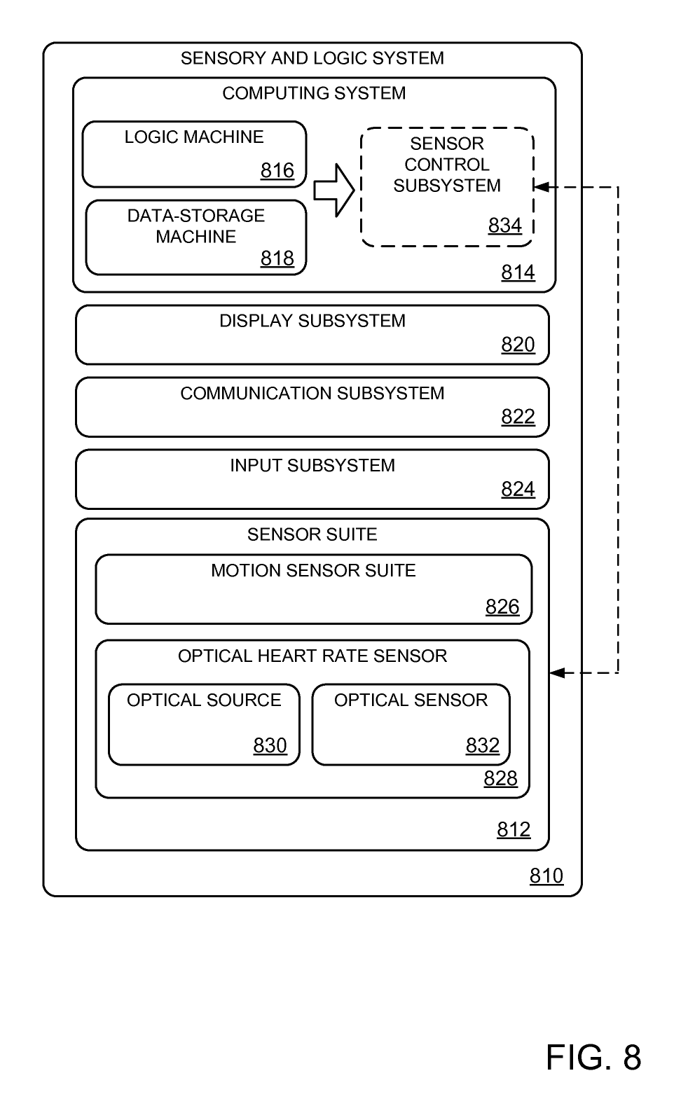

In wearable computing device 10, computing system 12 is situated below display 20 and operatively coupled to the display, along with loudspeaker 22, communication suite 24, and the various sensors. The computing system 12 includes a data-storage machine 27 to hold data and instructions, and a logic machine 28 to execute the instructions. Aspects of the computing system 12 are described in further detail with reference to FIGS. 2 and 8.

Display 20 may be any suitable type of display. In some configurations, a thin, low-power light emitting diode (LED) array or a liquid-crystal display (LCD) array may be used. An LCD array may be backlit in some implementations. In other implementations, a reflective LCD array (e.g., a liquid crystal on silicon, LCOS array) may be front lit via ambient light. A curved display may also be used. Further, AMOLED displays or quantum dot displays may be used.

Communication suite 24 may include any appropriate wired or wireless communications componentry. In FIGS. 1A and 1B, the communications suite includes USB port 30, which may be used for exchanging data between wearable computing device 10 and other computer systems, as well as providing recharge power. The communication suite may further include two-way Bluetooth, Wi-Fi, cellular, near-field communication, and/or other radios. In some implementations, the communication suite may include an additional transceiver for optical, line-of-sight (e.g., infrared) communication.

In wearable computing device 10, touch-screen sensor 32 is coupled to display 20 and configured to receive touch input from the user. The touch-screen sensor 32 may be resistive, capacitive, or optically based. Pushbutton sensors may be used to detect the state of push buttons 34, which may include rockers. Input from the pushbutton sensors may be used to enact a home-key or on-off feature, control audio volume, turn the microphone on or off, or other function.

FIGS. 1A and 1B show various other sensors of wearable computing device 10. Such sensors include microphone 36, visible-light sensor 38, ultraviolet sensor 40, and ambient temperature sensor 42. The microphone 36 provides input to computing system 12 that may be used to measure the ambient sound level or receive voice commands from the wearer. Input from the visible-light sensor, ultraviolet sensor, and ambient temperature sensor may be used to assess aspects of the wearer's environment—i.e., the temperature, overall lighting level, and whether the wearer is indoors or outdoors.

FIGS. 1A and 1B show a pair of contact sensor modules 44A and 44B, which contact the wearer's skin when wearable computing device 10 is worn. The contact sensor modules may include independent or cooperating sensor elements, to provide a plurality of sensory functions. For example, the contact sensor modules may provide an electrical resistance and/or capacitance sensory function, which measures the electrical resistance and/or capacitance of the wearer's skin. Computing system 12 may use such input to assess whether or not the device is being worn, for instance. In some implementations, the sensory function may be used to determine how tightly the wearable computing device is being worn. In the illustrated configuration, the separation between the two contact-sensor modules provides a relatively long electrical path length, for more accurate measurement of skin resistance. In some examples, a contact sensor module may also provide measurement of the wearer's skin temperature.

Arranged inside contact sensor module 44A in the illustrated configuration is the optical heart rate sensor 46. The optical heart rate sensor may include an optical source and matched optical sensor to detect blood flow through the capillaries in the skin and thereby provide a measurement of the wearer's heart rate, blood oxygen level, blood glucose level, or other biomarkers with optical properties.

In some examples, the computing system 12, via the sensory functions described herein, may be configured to acquire various forms of information about the wearer of wearable computing device 10. Such information would be acquired and used with utmost respect for the wearer's privacy. Accordingly, the sensory functions may be enacted subject to opt-in participation of the wearer. In implementations where personal data is collected on the device and transmitted to a remote system for processing, that data may be anonymized. In other examples, personal data may be confined to the wearable computing device, and only non-personal, summary data transmitted to the remote system.

FIG. 2 shows a schematic depiction of an example sensory-and-logic system 100 including a computing system 118 and example sensors that may be included in the wearable computing device of FIGS. 1A and 1B. The computing system 118 comprises a driver module 111 that may provide control signals to an optical heart rate sensor 146, including an optical source 103 and optical sensor 105, GPS receiver 154 and inertial measuring unit (IMU) 147. Control signals sent to the sensors may be based on signals received from these sensors and/or one or more other sensors, such as ambient light sensors, information stored in computing system 118, input signals, user settings, clock signals, and other signals. The driver module 11 also may sample time-dependent data 158 outputted by a sensor and provide such sampled data to one or more time-independent, sample-count-based calculation modules 160.

In some examples and as described in more detail below, the driver module 111 may down-sample the time-dependent data 158 outputted by a sensor at a time-dependent down-sampled frequency that is different from the time-dependent sensor frequency at which the sensor is natively operating. In one example, the IMU 147 may output time-dependent 6DOF accelerations at a time-dependent IMU frequency. As described in more detail below, under certain conditions the driver module 111 may down-sample the time-dependent 6DOF accelerations at a time-dependent down-sample IMU frequency that is less than the time-dependent IMU frequency.

Computing system 118 may receive raw signals from one or more of these sensors. Such raw signals may take the form of time-dependent data 158 that is outputted from a sensor at a time-dependent frequency. Each time-dependent datum may specify a parameter sensed by the corresponding sensor. For example, the heart rate sensor 146 may output time-dependent data 158 in the form of heart beat signals specifying a heart-beat at a time-dependent heart rate sensor frequency. The GPS receiver 154 may output time-dependent data 158 in the form of location signals specifying location and/or velocity at a time-dependent GPS frequency. The IMU 147 may output time-dependent data 158 in the form of 6DOF accelerations at a time-dependent IMU frequency specifying movement of the IMU.

As described in more detail below, one or more time-independent, sample-count-based calculation modules 160 may process the raw signals received from the sensors to determine and output various time-agnostic data 164 such as, for example, heart rate, caloric expenditure, steps taken, etc. For example, a time-independent, sample-count-based calculation module 160 may be configured to output a new time-agnostic datum 164 after receiving a specific number of the time-dependent data 158, regardless of an elapsed time taken to receive the specific number of time-dependent data. As described in more detail below, in some examples the new time agnostic-data 164 may comprise a biometric metric, such as a heart rate, number of steps taken, calories expended, distance traveled, speed traveled, etc.

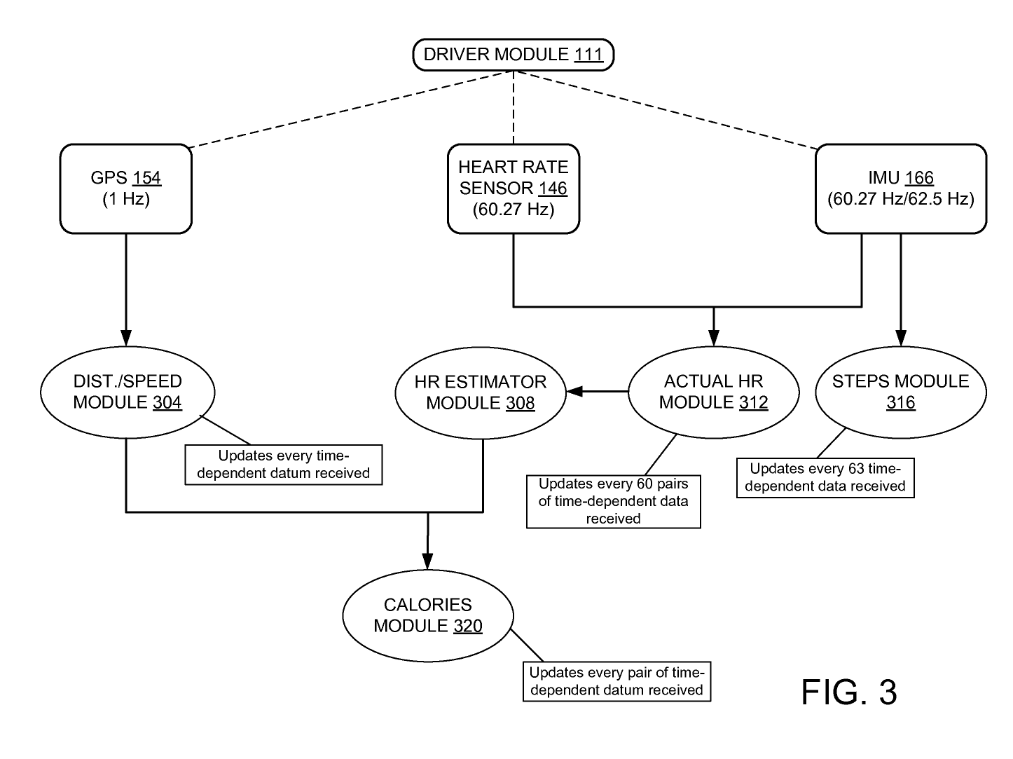

FIG. 3 shows one example of a driver module, sensors, sample-count-based calculation modules and a corresponding data flow in a computing device, such as wearable computing device 10. In the example of FIG. 3, the sensors include a time-dependent GPS receiver 154, time-dependent heart rate sensor 146 and time-dependent IMU 166. Each of the sensors may utilize a real-time clock to manage data delivery rates to the various sample-count-based calculation modules. It will be appreciated that in other examples, fewer or more time-dependent sensors may be provided.

In the example of FIG. 3, the time-independent, sample-count-based calculation modules include a distance/speed module 304, a heart rate estimator module 308, an actual heart rate module 312, a steps module 316 and a calories module 320. In this example the distance/speed module 304 may receive time-dependent location data that are outputted from the GPS device 154 at a time-dependent GPS frequency. The actual heart rate module 312 may receive time-dependent heart rate data that are outputted from the heart rate sensor 146 at a time-dependent heart rate sensor frequency, and may receive time-dependent 6DOF accelerations that are outputted from the IMU 166 at a first time-dependent IMU frequency.

The steps module 316 also may receive time-dependent 6DOF accelerations that are outputted from the IMU 166 at a second time-dependent IMU frequency that may be the same as or different from the first time-dependent IMU frequency. The calories module 320 may receive distance and speed data from the distance/speed module 304, and may receive heart rate data from the heart rate estimator module 308. In this example, the heart rate estimator module 308 may receive from the actual heart rate module 312 a time-agnostic datum in the form of a heart rate, such as a number of beats per minute or any other suitable measurement of heart rate. The heart rate estimator module 308 may pass the heart rate data unchanged to the calories module 320.

In the example of FIG. 3, both the actual heart rate module 312 and the steps module 316 are configured to receive time-dependent acceleration data outputted by the IMU 166. In this example, the actual heart rate module 312 and the steps module 316 are configured to update their respective time-agnostic data after receiving different specific numbers of acceleration data. For example, the steps module 316 may be configured to update its number of steps datum after receiving 63 acceleration data. The actual heart rate module 312 may be configured to update its heart rate datum after receiving 60 acceleration data and combining this data with heart rate data from heart rate sensor 146, as described in more detail below.

Each of the modules may be configured to output a new time-agnostic datum after receiving a specific number of time-dependent data regardless of the elapsed time taken to receive the specific number of time-dependent data. A module may be tuned to update and output its time-agnostic data after receiving any specific number of time-dependent data. More specifically, a time-independent sample-count-based calculation module may be tuned to produce outputs at a desired rate that is in proportion to an expected input rate. For example, if a downstream module expects one output per second, and the time-independent sample-count-based calculation module expects to receive inputs 63 times per second, the time-independent sample-count-based calculation module can be tuned to produce an output after receiving 63 inputs. If time-dependent data are fed to the time-independent sample-count-based calculation module at a constant rate (e.g., 63 per second), the time-independent sample-count-based calculation module will output time-agnostic data at a constant rate (e.g., 1 per second). However, if the input rate is changed, the output rate will change accordingly, because the output rate is dictated only by the number of received inputs, not by an elapsed time.

As noted above, the actual heart rate module 312 may be configured to output a new time-agnostic heart rate after receiving 60 acceleration data and combining this data with 60 heart rate data from heart rate sensor 146. In this example, the driver module 111 may sample the heart rate sensor 146 at a heart rate sensor frequency of 60.27 Hz. To account for variations in heart rate sensor data due to accelerations of a user's hand, the heart rate sensor data may be synchronized with the acceleration data from the IMU 166, and pairs of synchronized heart rate data and acceleration data may be provided to the actual heart rate module 312. Using this paired data, the actual heart rate module 312 may calculate and output a new heart rate datum after receiving 60 pairs of such data.

Such heart rate data may be provided to the heart rate estimator module 308, which may pass such data to the calories module 320. The distance/speed module may be configured to output a new time-agnostic distance and/or speed datum after receiving each time-dependent location datum from the GPS 154, regardless of the elapsed time taken to receive each datum. In this example, the GPS 154 may output a location datum at a GPS frequency of 1 Hz.

The calories module 320 may be configured to determine a calories expended datum based on the heart rate data received from the heart rate estimator module 308 and the distance and/or speed data received from the distance/speed module 304. The calories module 320 may be configured to update and output a calories expended datum for each pair of heart rate datum and distance and/or speed datum received.

It will be appreciated that because the modules are configured to be time-independent and sample-count-based, any suitable update rate for a module may be achieved by changing the input rate of the time-dependent data into a module. Accordingly, a module may be configured to dynamically change its update rate in proportion to an input rate of the time-dependent data. For example, the output frequency and/or sample rate of one or more of the time-dependent sensors may be increased to effectively increase the update rate for a module.

In some examples, a module may receive time-dependent data at a time-dependent frequency that is greater than an original time-dependent frequency at which the data was collected. For example, a module may be communicatively coupled to a data storage machine that may provide time-dependent data to the module at a time-dependent data transfer rate.

In one example, 6DOF acceleration data may be collected over a one year period at an IMU frequency of 62.5 Hz., and may be stored in a data storage machine. Such data may be provided by the data storage machine to the steps module 316 via a network, computer bus, or other suitable communication interface at a much higher data transfer rate, such as 10 Mbit/sec, 100 Mbit/sec, 32 Gbit/sec or other suitable data transfer rate. In this manner, and because the steps module 316 is a time-independent, sample-count-based calculation module, data throughput of the module may be significantly increased as compared to the time-dependent frequency at which the data was collected. In this manner for example, validating a results accuracy of the steps module 316 over the one year data collection period may be accomplished quickly and efficiently.

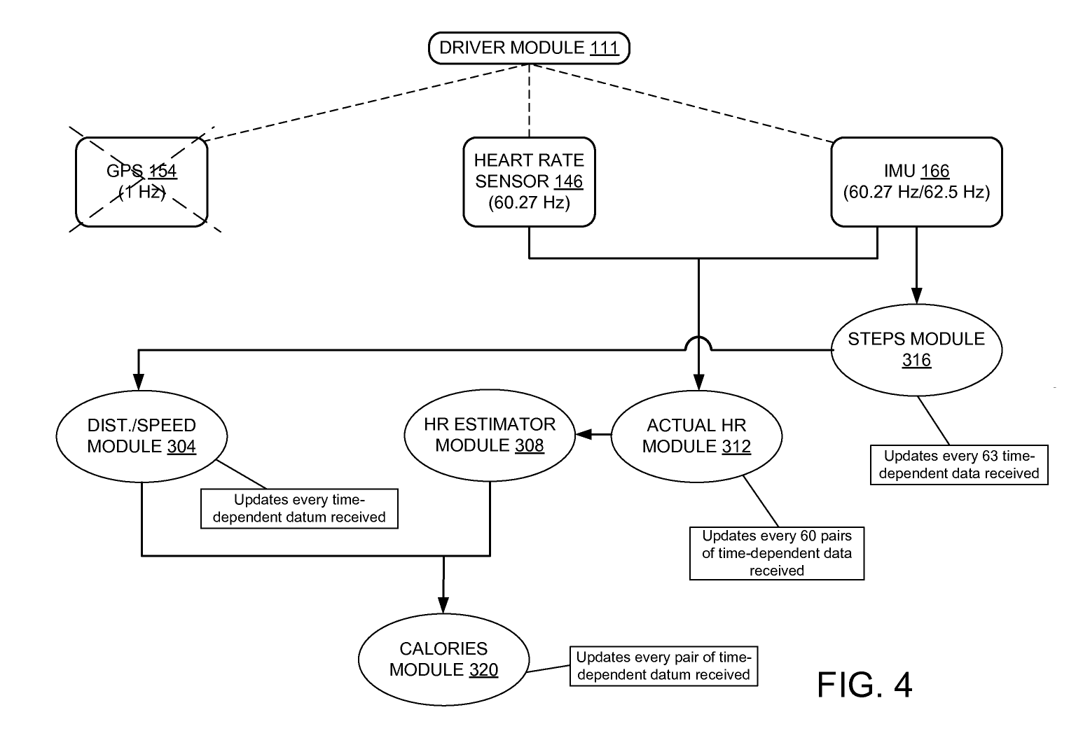

FIG. 4 shows another example use case and data flow among the driver module, sensors, and sample-count-based calculation modules of FIG. 3. In the example of FIG. 4, operation of the GPS 154 has been ceased to, for example, reduce power consumption of the wearable computing device 10. Accordingly, the distance/speed module 304 may be configured to receive the number of steps data outputted from the steps module 316. As in the example of FIG. 3, the steps module 316 may update its number of steps datum upon receiving each set of 63 time-dependent location data from the IMU 166. The distance speed/module 304 may update its distance and/or speed datum upon receiving each number of steps datum from the steps module 316.

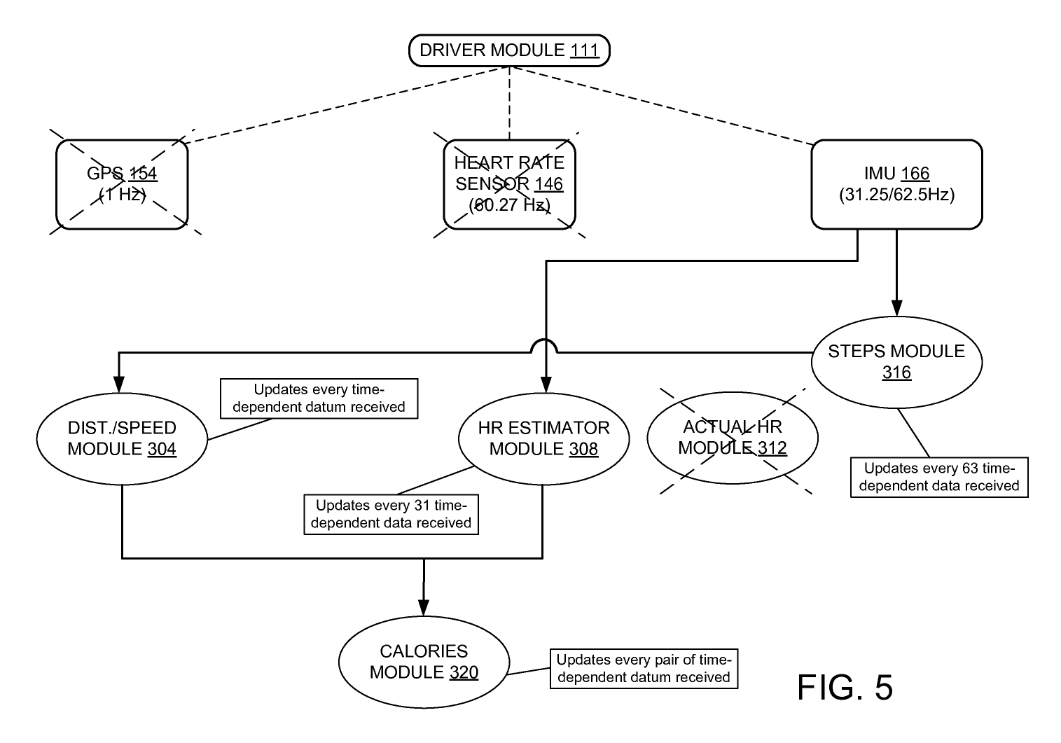

FIG. 5 shows another example use case and data flow among the driver module, sensors, and sample-count-based calculation modules of FIG. 3. In the example of FIG. 5, operation of the GPS 154 and the heart rate sensor 146 have been ceased to, for example, reduce power consumption of the wearable computing device 10. In one example, when the wearable computing device 10 is operating in a daily mode, the heart rate sensor 146 may be controlled to operate for 1 minute during every 10 minute period.

As shown in the examples of FIGS. 3 and 4, when the heart rate sensor 146 is operating and providing output, the actual heart rate module 312 may receive accelerations from the IMU 166 that is operating at or being sampled at a frequency of 60.27 Hz. In the example of FIG. 5, because the heart rate sensor 146 is not providing output, the actual heart rate module 312 may not be used to determine heart rate data. Accordingly, the heart rate estimator module 308 may be utilized to calculate heart rate data using accelerations received from the IMU 166.

In some examples the heart rate estimator module 308 may comprise a learning algorithm that maps an acceleration to a heart rate number. The heart rate estimator module 308 may utilize heart rate data from the heart rate sensor 146 to adaptively calibrate when the heart rate sensor is operating.

In some examples, the heart rate estimator module 308 may provide an acceptable degree of accuracy when receiving acceleration data at a time-dependent frequency that is less than the frequency at which the steps module 316 receives acceleration data. Accordingly and in response to output from the heart rate sensor 146 ceasing, the driver module 111 may dynamically down-sample the acceleration data outputted from the IMU 166 at a time-dependent down-sample frequency that is less than the frequency at which the steps module 316 receives acceleration data.

In the example of FIG. 5, the driver module 111 may dynamically down-sample and deliver the acceleration data outputted from the IMU 166 at 31.25 Hz to the heart rate estimator module 308. In this example, the heart rate estimator module may be configured to output a new time-agnostic heart rate datum after receiving 31 accelerations from the IMU 166. Accordingly and in this example, because the heart rate sensor 146 is operating for 1 minute during every 10 minute period, the acceleration data from the IMU 166 will be dynamically down-sampled to 31.25 Hz. and delivered to the heart rate estimator module 308 for 9 minutes of every 10 minute period.

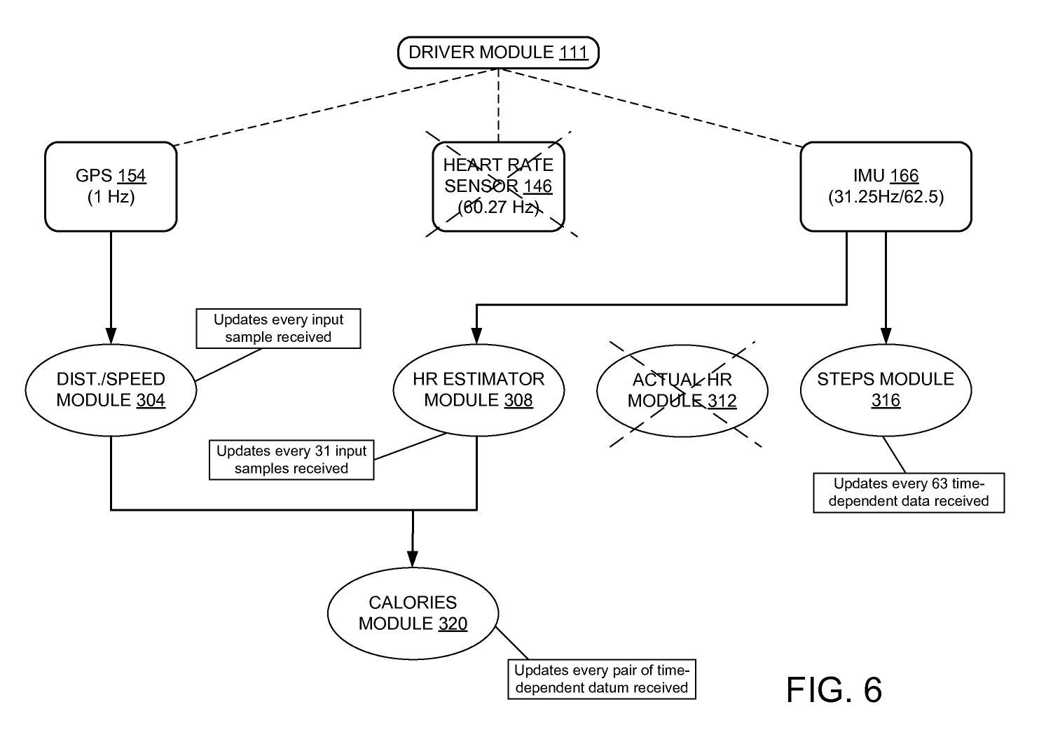

FIG. 6 shows another example use case and data flow among the driver module, sensors, sample-count-based calculation modules of FIG. 3 in which the heart rate sensor 146 is not operating. In the example of FIG. 6, the GPS 154 and IMU 166 are operating and outputting their time-dependent data, while operation of the heart rate sensor 146 has been ceased. As in the example of FIG. 5, in this example the heart rate estimator module 308 may be utilized to calculate heart rate data using accelerations received from the IMU 166.

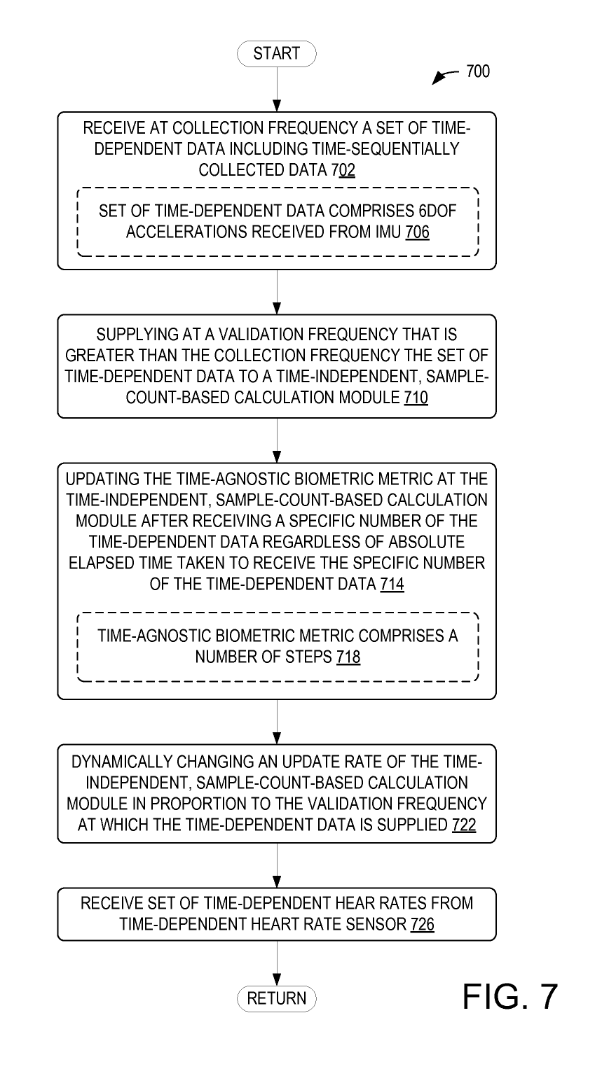

FIG. 7 shows a flow chart of an example method 700 for updating a time-agnostic biometric metric. For example, method 700 may be performed by any suitable computing device, such as wearable computing device 10. In some examples, some portions of the method 700 may be performed by one or more devices while other portions of the method 700 may be performed by one or more other devices. At 702, method 700 may include receiving at a collection frequency a set of time-dependent data including a plurality of time-sequentially collected data. At 706 the set of time-dependent data may comprise 6DOF accelerations that are received from a time-dependent IMU. For example, time-dependent motion data may be continuously received from an IMU at a collection frequency of 62.5 Hz. over a 24-hour period. In some examples, the motion data may be received by a storage machine located in the wearable computing device 10 or in another computing device.

At 710 the method 700 may include supplying at a validation frequency that is greater than the collection frequency the set of time-dependent data to a time-independent, sample-count-based calculation module. For example and as noted above, the validation frequency may correspond to a network or computer bus data transfer rate, such as 10 Mb/sec, 100 Mb/sec, 32 Gbit/sec or other suitable data transfer rate.

At 714 the method may include updating the time-agnostic biometric metric at the time-independent, sample-count-based calculation module after receiving a specific number of the time-dependent data regardless of elapsed time taken to receive the specific number of the time-dependent data. In the present example, the time-independent, sample-count-based calculation module may update the time-agnostic biometric metric after receiving 126 time-dependent data.

In one example where time-dependent motion data is continuously received by the time-independent, sample-count-based calculation module from an IMU at a collection frequency of 62.5 Hz. over a 24-hour period, the module may update the time-agnostic biometric metric after receiving each set of 126 time-dependent data over the 24-hour period. In one example where each acceleration datum comprises a 14-bit datum, over the 24-hour period the module may receive approximately 76 Mb of data.

In another example, the 76 Mb of time-dependent acceleration data may be stored in a storage machine. The storage machine may supply the data to the time-independent, sample-count-based calculation module at a validation frequency of 100 Mb/sec. For example and as noted above, the validation frequency may correspond to a network or computer bus data transfer rate, such as Mbit/sec, 100 Mbit/sec, 32 Gbit/sec or other suitable data transfer rate. In this manner, because the module is time-independent and sample-count-based, the module may update the time-agnostic biometric metric after receiving each set of 126 time-dependent data. Accordingly and at a data transfer rate of 100 Mb/sec, the 76 Mb of data may be processed by the module substantially faster as compared to receiving the data over the 24-hour period.

At 718 the time-agnostic biometric metric comprises a number of steps. At 722 the method 700 may include dynamically changing an update rate of the time-independent, sample-count-based calculation module in proportion to the validation frequency at which the time-dependent data is supplied. With reference to the examples discussed above, in some examples a module may first receive time-dependent data at a first validation frequency corresponding to a frequency of output from a sensor.

Subsequently the module may receive time-dependent data at a second validation frequency corresponding to a data transfer rate of a network. Accordingly, upon the module receiving the data at the second validation frequency, the driver module 111 may dynamically change the update rate of the module in proportion to the second validation frequency at which the data is supplied. At 726 the method 700 may include receiving a set of time-dependent heart rates from a time-dependent heart rate sensor.

In some implementations and as evident from the foregoing description, the methods and processes described herein may be tied to a sensory-and-logic system of one or more machines. Such methods and processes may be implemented as a computer-application program or service, an application-programming interface (API), a library, firmware, and/or other computer-program product. FIGS. 1A, 1B and 2 show non-limiting examples of a sensory-and-logic system to enact the methods and processes described herein. However, these methods and process may also be enacted on sensory-and-logic systems of other configurations and form factors, as shown schematically in FIG. 8.

FIG. 8 schematically shows a form-agnostic sensory-and-logic system 810 that includes a sensor suite 812 operatively coupled to a computing system 814. The computing system includes a logic machine 816 and a data-storage machine 818. The computing system is operatively coupled to a display subsystem 820, a communication subsystem 822, an input subsystem 824, and/or other components not shown in FIG. 8.

Logic machine 816 includes one or more physical devices configured to execute instructions. The logic machine may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

Logic machine 816 may include one or more processors configured to execute software instructions. Additionally or alternatively, the logic machine may include one or more hardware or firmware logic machines configured to execute hardware or firmware instructions. Processors of the logic machine may be single-core or multi-core, and the instructions executed thereon may be configured for sequential, parallel, and/or distributed processing. Individual components of a logic machine optionally may be distributed among two or more separate devices, which may be remotely located and/or configured for coordinated processing. Aspects of a logic machine may be virtualized and executed by remotely accessible, networked computing devices in a cloud-computing configuration.

Data-storage machine 818 includes one or more physical devices configured to hold instructions executable by logic machine 816 to implement the methods and processes described herein. When such methods and processes are implemented, the state of the data-storage machine may be transformed—e.g., to hold different data. The data-storage machine may include removable and/or built-in devices; it may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. The data-storage machine may include volatile, nonvolatile, dynamic, static, read/write, read-only, random-access, sequential-access, location-addressable, file-addressable, and/or content-addressable devices.

It will be appreciated that data-storage machine 818 includes one or more physical devices. However, aspects of the instructions described herein alternatively may be propagated by a communication medium (e.g., an electromagnetic signal, an optical signal, etc.) that is not held by a physical device for a finite duration.

Software modules or programs may be implemented to perform one or more particular functions. In some cases, such a module or program may be instantiated via logic machine 816 executing instructions held by data storage machine 818. It is to be understood that different modules or programs may be instantiated from the same application, service, code block, object, library, routine, API, function, etc. Likewise, the same module or program may be instantiated by different applications, services, code blocks, objects, routines, APIs, functions, etc. The terms “module” and “program” are meant to encompass individual or groups of executable files, data files, libraries, drivers, scripts, database records, etc.

Aspects of logic machine 816 and data-storage machine 818 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

Display subsystem 820 may be used to present a visual representation of data held by data-storage machine 818. This visual representation may take the form of a graphical user interface (GUI). As the herein described methods and processes change the data held by the storage machine, and thus transform the state of the storage machine, the state of display subsystem 820 may likewise be transformed to visually represent changes in the underlying data. Display subsystem 820 may include one or more display subsystem devices utilizing virtually any type of technology. Such display subsystem devices may be combined with logic machine 816 and/or data-storage machine 818 in a shared enclosure, or such display subsystem devices may be peripheral display subsystem devices. Display 20 of FIGS. 1A and 1B is an example of display subsystem 820.

Communication subsystem 822 may be configured to communicatively couple computing system 814 to one or more other computing devices. The communication subsystem may include wired and/or wireless communication devices compatible with one or more different communication protocols. As non-limiting examples, the communication subsystem may be configured for communication via a wireless telephone network, a local- or wide-area network, and/or the Internet. Communication suite 24 of FIGS. 1A and 1B is an example of communication subsystem 822.

Input subsystem 824 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity. Touch-screen sensor 32 and push buttons 34 of FIGS. 1A and 1B are examples of input subsystem 824.

Sensor suite 812 may include one or more different sensors—e.g., a touch-screen sensor, push-button sensor, microphone, visible-light sensor, ultraviolet sensor, ambient-temperature sensor, contact sensors, and/or GPS receiver—as described above with reference to FIGS. 1A and 1B. Sensor suite 812 may include motion sensor suite 826. Motion sensor suite 826 may include an inertial measuring unit comprising one or more of an accelerometer, gyroscope, magnetometer, or other suitable motion detectors.

Sensor suite 812 may further include optical heart rate sensor 828. As described herein, optical heart rate sensor 828 may include optical source 830 and optical sensor 832. Optical source 830 may comprise one or more LED emitters, for example, while optical sensor 832 may comprise one or more photodiodes matched to detect light at frequencies that are based on the frequencies of light output by the optical source. Optical source 830 may be configured to illuminate blood vessels through the skin of the user, and optical sensor 832 may be configured to measure illumination reflected from or transmitted through blood vessels.

Computing system 814 may include a sensor control subsystem 834 which may be communicatively coupled to logic machine 816 and data-storage machine 818. Sensor control subsystem 834 may provide control signals to one or more sensors of the sensor suite 812 and/or may sample data outputted by such sensors. Sensory control subsystem 834 also may comprise one or more driver modules, such as driver module 111.

Sensor control subsystem 834 may receive raw signals from one or more sensors of the sensor suite 812, and may further process the raw signals to determine heart rate, caloric expenditures, etc. Sensor control subsystem 834 may include the time-independent, sample-count-based calculation modules discussed above. Processed signals may be stored and output via computing system 814. Control signals sent to sensors of the sensor suite 812 may be based on signals received or derived from sensor suite 812, information stored in data-storage machine 818, input received from communication subsystem 822, input received from input subsystem 824, etc.

In another example implementation, a computing device comprises a time-dependent sensor configured to output a time-dependent datum at a time-dependent frequency, the time-dependent datum specifying a sensed parameter; and a time-independent, sample-count-based calculation module configured to output a new time-agnostic datum after receiving a specific number of the time-dependent data regardless of elapsed time taken to receive the specific number of the time-dependent data. In one example implementation that optionally may be combined with any of the features described herein, the time-dependent sensor comprises an inertial measuring unit. In one example implementation that optionally may be combined with any of the features described herein, the computing device further comprises a driver module configured to dynamically down-sample the time-dependent data at a time-dependent down-sample frequency, and to provide the down-sampled data to the time-independent, sample-count-based calculation module. In one example implementation that optionally may be combined with any of the features described herein, the new time-agnostic datum comprises a biometric metric. In one example implementation that optionally may be combined with any of the features described herein, the biometric metric comprises a heart rate. In one example implementation that optionally may be combined with any of the features described herein, the biometric metric comprises a number of steps. In one example implementation that optionally may be combined with any of the features described herein, wherein the time-dependent sensor is an inertial measuring unit (IMU), the time-dependent datum is a 6DOF acceleration, the time-dependent frequency is a time-dependent IMU frequency, and the sensed parameter is movement of the inertial measuring unit, the computing device comprises a time-dependent heart rate sensor configured to output a time-dependent heart rate datum at a time-dependent heart rate sensor frequency. In one example implementation that optionally may be combined with any of the features described herein, in response to output from the time-dependent heart rate sensor ceasing, the output from the IMU is dynamically down-sampled at a time-dependent down-sample frequency that is less than the time-dependent IMU frequency.

In another example implementation, a wearable computing device comprises a time-dependent inertial measuring unit (IMU) configured to output a time-dependent 6DOF acceleration at a time-dependent IMU frequency; and a time-independent, sample-count-based calculation module configured to output a new time-agnostic biometric metric after receiving a specific number of the time-dependent 6DOF accelerations regardless of elapsed time taken to receive the specific number of the time-dependent 6DOF accelerations. In one example implementation that optionally may be combined with any of the features described herein, the wearable computing device comprises a driver module configured to dynamically down-sample the time-dependent 6DOF accelerations at a time-dependent down-sample frequency, and to provide the down-sampled time-dependent 6DOF accelerations to the time-independent, sample-count-based calculation module. In one example implementation that optionally may be combined with any of the features described herein, the new time-agnostic biometric metric comprises a heart rate. In one example implementation that optionally may be combined with any of the features described herein, the new time-agnostic biometric metric comprises a number of steps. In one example implementation that optionally may be combined with any of the features described herein, the wearable computing device comprises a time-dependent heart rate sensor configured to output a time-dependent heart rate datum at a time-dependent heart rate sensor frequency. In one example implementation that optionally may be combined with any of the features described herein, in response to output from the time-dependent heart rate sensor ceasing, the output from the IMU is dynamically down-sampled at a time-dependent down-sample frequency that is less than the time-dependent IMU frequency. In one example implementation that optionally may be combined with any of the features described herein, the time-dependent 6DOF accelerations are sampled at the time-dependent IMU frequency for a first period of time during which the heart rate sensor is operating, and the time-dependent 6DOF accelerations are dynamically down-sampled at the time-dependent down-sample frequency for a second period of time during which the heart rate sensor is not operating, where the second period of time is longer than the first period of time.

In another example implementation, a method of updating a time-agnostic biometric metric comprises receiving at a collection frequency a set of time-dependent data including a plurality of time-sequentially collected data; supplying at a validation frequency that is greater than the collection frequency the set of time-dependent data to a time-independent, sample-count-based calculation module; and updating the time-agnostic biometric metric at the time-independent, sample-count-based calculation module after receiving a specific number of the time-dependent data regardless of elapsed time taken to receive the specific number of the time-dependent data. In one example implementation that optionally may be combined with any of the features described herein, the method comprises dynamically changing an update rate of the time-independent, sample-count-based calculation module in proportion to the validation frequency at which the time-dependent data is supplied. In one example implementation that optionally may be combined with any of the features described herein, the set of time-dependent data comprises 6DOF accelerations received from a time-dependent inertial measuring unit. In one example implementation that optionally may be combined with any of the features described herein, the method comprises receiving a set of time-dependent heart rates from a time-dependent heart rate sensor. In one example implementation that optionally may be combined with any of the features described herein, the time-agnostic biometric metric comprises a number of steps.

It will be understood that the configurations and approaches described herein are exemplary in nature, and that these specific implementations or examples are not to be taken in a limiting sense, because numerous variations are feasible. The specific routines or methods described herein may represent one or more processing strategies. As such, various acts shown or described may be performed in the sequence shown or described, in other sequences, in parallel, or omitted.

The subject matter of this disclosure includes all novel and non-obvious combinations and sub-combinations of the various processes, systems and configurations, and other features, functions, acts, and/or properties disclosed herein, as well as any and all equivalents thereof.

1. A computing device, comprising:
a time-dependent sensor configured to output a time-dependent datum at a time-dependent frequency, the time-dependent datum specifying a sensed parameter; and
a time-independent, sample-count-based calculation module configured to output a new time-agnostic datum after receiving a specific number of the time-dependent data regardless of elapsed time taken to receive the specific number of the time-dependent data.

2. The computing device of claim 1, wherein the time-dependent sensor comprises an inertial measuring unit.

3. The computing device of claim 1, further comprising a driver module configured to dynamically down-sample the time-dependent data at a time-dependent down-sample frequency, and to provide the down-sampled data to the time-independent, sample-count-based calculation module.

4. The computing device of claim 1, wherein the new time-agnostic datum comprises a biometric metric.

5. The computing device of claim 4, wherein the biometric metric comprises a heart rate.

6. The computing device of claim 4, wherein the biometric metric comprises a number of steps.

7. The computing device of claim 1, wherein the time-dependent sensor is an inertial measuring unit (IMU), the time-dependent datum is a 6DOF acceleration, the time-dependent frequency is a time-dependent IMU frequency, and the sensed parameter is movement of the inertial measuring unit, further comprising a time-dependent heart rate sensor configured to output a time-dependent heart rate datum at a time-dependent heart rate sensor frequency.

8. The computing device of claim 7, wherein in response to output from the time-dependent heart rate sensor ceasing, the output from the IMU is dynamically down-sampled at a time-dependent down-sample frequency that is less than the time-dependent IMU frequency.

9. A wearable computing device, comprising:
a time-dependent inertial measuring unit (IMU) configured to output a time-dependent 6DOF acceleration at a time-dependent IMU frequency;
a time-independent, sample-count-based calculation module configured to output a new time-agnostic biometric metric after receiving a specific number of the time-dependent 6DOF accelerations regardless of elapsed time taken to receive the specific number of the time-dependent 6DOF accelerations.

10. The wearable computing device of claim 9, further comprising a driver module configured to dynamically down-sample the time-dependent 6DOF accelerations at a time-dependent down-sample frequency, and to provide the down-sampled time-dependent 6DOF accelerations to the time-independent, sample-count-based calculation module.

11. The wearable computing device of claim 9, wherein the new time-agnostic biometric metric comprises a heart rate.

12. The wearable computing device of claim 9, wherein the new time-agnostic biometric metric comprises a number of steps.

13. The wearable computing device of claim 9, further comprising a time-dependent heart rate sensor configured to output a time-dependent heart rate datum at a time-dependent heart rate sensor frequency.

14. The wearable computing device of claim 13, wherein in response to output from the time-dependent heart rate sensor ceasing, the output from the IMU is dynamically down-sampled at a time-dependent down-sample frequency that is less than the time-dependent IMU frequency.

15. The wearable computing device of claim 14, wherein the time-dependent 6DOF accelerations are sampled at the time-dependent IMU frequency for a first period of time during which the heart rate sensor is operating, and the time-dependent 6DOF accelerations are dynamically down-sampled at the time-dependent down-sample frequency for a second period of time during which the heart rate sensor is not operating, where the second period of time is longer than the first period of time.

16. A method of updating a time-agnostic biometric metric, the method comprising:
receiving at a collection frequency a set of time-dependent data including a plurality of time-sequentially collected data;
supplying at a validation frequency that is greater than the collection frequency the set of time-dependent data to a time-independent, sample-count-based calculation module; and
updating the time-agnostic biometric metric at the time-independent, sample-count-based calculation module after receiving a specific number of the time-dependent data regardless of elapsed time taken to receive the specific number of the time-dependent data.

17. The method of claim 16, further comprising dynamically changing an update rate of the time-independent, sample-count-based calculation module in proportion to the validation frequency at which the time-dependent data is supplied.

18. The method of claim 16, wherein the set of time-dependent data comprises 6DOF accelerations received from a time-dependent inertial measuring unit.

19. The method of claim 18, further comprising receiving a set of time-dependent heart rates from a time-dependent heart rate sensor.

20. The method of claim 16, wherein the time-agnostic biometric metric comprises a number of steps.
